(19) United States
(12) Patent Application Publication
(10) Pub. No.: US 2010/0221351 A1
(43) Pub. Date: Sep. 2, 2010

(54) Controlled Drug Delivery Using a Thermally Responsive Nanocapsule to Augment Cryoablation

(75) Inventor: Xiaoming He, Lexington, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/714,931

(22) Filed: Mar. 1, 2010

Related U.S. Application Data
(60) Provisional application No. 61/208,724, filed on Feb. 27, 2009.

(51) Int. Cl. A61K 9/51; A61K 31/337; A61K 31/407; A61K 38/10; A61K 31/473; A61K 31/4745; A61K 31/7048; A61K 31/437; A61K 31/165; A61K 31/704; A61K 31/122; A61K 31/136; A61K 38/12; A61K 31/568; A61K 31/56; A61K 31/245; A61K 31/167; A61K 31/138; A61K 31/7064; A61P 35/00

(52) U.S. Cl. 424/493; 514/449; 514/411; 514/14; 514/298; 514/294; 514/27; 514/283; 514/629; 514/34; 514/680; 514/656; 514/33; 514/10; 514/178; 514/181; 514/535; 514/626; 514/652; 514/46; 424/497; 424/490; 977/906; 977/788; 977/795; 977/833

(57) ABSTRACT

In accordance with certain embodiments of the present disclosure, A method for intracellular delivery of cytotoxin in combination with cyoablation is provided. The method includes encapsulation of one or more cytotoxins in a thermally responsive nanocapsule by decreasing the temperature of the nanocapsule to increase the permeability of the nanocapsule whereby the one or more cytotoxins are sucked into or diffuse into the nanocapsule. The temperature of the nanocapsule is increased and the nanocapsule is delivered into a cell. Cryoablation is performed in proximity to the cell resulting in the release of the one or more cytotoxins from the nanocapsule into the cell.

### CROSS-REFERENCE TO RELATED APPLICATION

The present application is based on and claims priority to U.S. Provisional Application 61/208,724 having a filing date of Feb. 27, 2009, which is incorporated by reference herein.

### BACKGROUND OF THE INVENTION

Cryoablation (or cryosurgery) is a minimally invasive surgical technique for destroying diseased tissue by lowering the tissue temperature to a threshold value, usually between −40 and −20° C. dependent on the tissue type. The frozen and thawed tissue is left in situ and is disposed by the body immune system. Cryoablation has been used to treat a variety of diseases including the early stage breast cancer with promising outcomes. A major advantage of cryoablation over other minimally invasive procedures using elevated temperature is that modern imaging technology such as ultrasound can be used to monitor intraoperatively the progressing edge of iceball containing frozen tissue. Unfortunately, the frozen tissue temperature (approximately −5° C.) at the edge of an iceball is usually much higher than that required for cell/tissue destruction (i.e., −40˜−20° C.), which makes it difficult to determine the real ‘kill zone’ based on intraoperative imaging of the iceball formed. This difficulty may result in incomplete killing and cancer recurrence. Therefore, enhancement of tissue destruction under mild freezing/thawing conditions like that near the edge of an iceball is needed.

A number of strategies have been proposed to augment cryodestruction of unwanted cells and tissue under mild freezing/thawing conditions. For example, it has been found that modification of the ice morphology by adding glycine (a nonessential amino acid) or an excess of electrolytes such as sodium and potassium chloride can significantly enhance cryoinjury in prostate and breast cancer cells in vitro. The antifreeze protein that is used by arctic fish to protect them from being frozen has been show to augment cryoinjury of frozen prostate cancer cells both in vitro and in vivo. A significant augmentation of cryoablation in vivo has also been achieved by preoperative injection of tumor necrosis factor-alpha (TNF-α) to induce pre-treatment inflammation in prostate cancer tissue. Enhanced suppression of tumor growth in patients has been observed by combining cryoablation with local injection of immupotentiator OK-423 (a streptococcal preparation) and mild systemic chemotherapy. In addition, anticancer drugs such as peplomycin, 5-fluorouracil, and bleomycin in their free form have been shown to significantly enhance cryoinjury in a number of cancer cell lines under mild freezing/thawing conditions. Of note, although the aforementioned chemical adjuvants can significantly enhance cryoinjury in cancer cells, they are generally toxic and may cause damage to healthy cells as well if delivered in the free form and in an uncontrolled manner in vivo¹⁴. Therefore, it is of great importance to explore the capability of administering a chemical adjuvant for augmenting cryoinjury in a controllable manner to minimize side effects in vivo.

### SUMMARY

In accordance with certain embodiments of the present disclosure, a method for intracellular delivery of cytotoxin in combination with cryoablation is provided. The method includes encapsulation of one or more cytotoxins in a thermally responsive nanocapsule by decreasing the temperature of the nanocapsule to increase the permeability of the nanocapsule whereby the one or more cytotoxins are sucked into or diffuse into the nanocapsule. The temperature of the nanocapsule is increased and the nanocapsule is delivered into a cell. Cryoablation is performed in proximity to the cell resulting in the release of the one or more cytotoxins from the nanocapsule into the cell.

In accordance with still other aspects of the present disclosure, a thermally responsive nanocapsule comprising is provided. The nanocapsule comprises a polymeric hydrogel nanocapsule which includes a shell and a core. The shell has a diameter of greater than 150 nm at a temperature of less than 25° C. and a diameter of less than 150 nm at a temperature of greater than 25° C. The core comprises one or more cytotoxins.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures in which:

FIG. 1. Temperature dependent diameter and surface charge of the nanocapsule synthesized: (A), data from dynamic light scattering (DLS) measurement and (B), a schematic demonstration of the thermal responsiveness.

FIG. 2. Release of trehalose (0.3 g) into 1 L water during dialysis under 3 conditions: free dissolved, nanoencapsulated (NE) with cooling/heating between 22 and 37° C. (NE22-37C), and nanoencapsulated kept at 37° C. (NE37C) before dialysis.

FIG. 3. Typical micrographs showing intracellular nanocapsule (green, A and D), endosome/lysosome (red, B and E), and the merged views (C for A&B and F for D&E): upper and bottom panels are for cells without and with cooling at 22° C., respectively.

FIG. 4. A schematic representation of the process for nanoencapsulation of doxorubicin.

FIG. 5. A schematic showing cellular uptake of the nanoencapsulated DOX and its controlled release by cooling and heating.

### DETAILED DESCRIPTION OF REPRESENTATIVE EMBODIMENTS

Reference will now be made in detail to embodiments of the disclosed subject matter, one or more examples of which are set forth below. Each example is provided by way of explanation of the subject matter, not limitation of the subject matter. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

In general, the present disclosure is directed to a novel, minimally invasive approach for cancer treatment that combines cryoablation with nanocapsule mediated intracellular delivery and controlled release of a cytotoxin. As a result of the thermal responsiveness of the nanocapsule, release of the cytotoxin can be controlled to be within the cancer cells to be destroyed by cryoablation.

Described herein are products and methods to integrate cryoablation with controlled drug delivery using a thermally responsive nanocapsule to achieve complete cancer destruction in the iceball and simultaneously minimize systemic drug cytotoxicity. Due to the small size and highly positively charged surface of the thermally responsive nanocapsule at about 37° C., drugs encapsulated in the nanocapsule can be specifically delivered to and taken up by cancer cells in tumor through intravenous injection (particularly when coated with polyethylene and moieties that have high affinity with a specific cancer cell). Moreover, drug release from the nanocapsule at about 37° C. is negligible as a result of its extremely low wall permeability at normal physiological temperature, which significantly reduces the drug systemic cytotoxicity. However, the double cooling/heating cycles between a subzero temperature and about 37° C. of a typical cryoablation procedure are particularly effective for drug release from the nanocapsule. This is because the volume expansion (>50 times) of the nanocapsule during cooling can suck water to dissolve the encapsulated drug and the dissolved drug can be squeezed out of the nanocapsule during heating as a result of its volume contraction (>50 times). Therefore, a significant drug release from the nanocapsule into the cytosol is only limited to the cancer cells with cryoablation, which can significantly augment cryoinjury to ensure complete destruction of the whole frozen tumor.

In this regard, cryoablation (or cryosurgery) is well known in the art. Cryoablation of tissues is a method of treatment for a variety of pathological conditions including malignancies in body organs such as the breast, prostate, kidney, liver, and other organs. Cryoablation of pathological tissues or other unwanted tissues is typically accomplished by utilizing imaging modalities, such as x-ray, ultrasound, CT, and MRI, to identify a locus for ablative treatment, then inserting one or more cryoprobes into that selected treatment locus, then cooling the treatment heads of the inserted cryoprobes sufficiently to cause the tissues surrounding the treatment heads to reach cryoablation temperatures, typically about −40° C., and allowing the tissues to thaw. Typical cryoablation procedures involve double cooling/heating cycles.

As described above, the present disclosure is directed to a minimally invasive approach for cancer treatment that combines cryoablation with nanocapsule mediated intracellular delivery and controlled release of a cytotoxin. As used herein, the term “cancer” unless otherwise indicated, refers to diseases that are characterized by uncontrolled, abnormal cell growth and/or proliferation. In one aspect of the invention, the cancer comprises a solid tumor including, but not limited to, metastatic solid tumors. In one aspect the solid tumor is an endothelial cell carcinoma, including, but not limited to, renal cell carcinoma, colon carcinoma, transitional cell carcinoma, lung carcinoma, breast carcinoma and prostatic carcinoma. Examples of renal cell carcinoma include, but are not limited to, clear cell carcinoma, papillary carcinoma, chromophobe carcinoma, collecting duct carcinoma and unclassified carcinoma. Examples of lung carcinoma include, but are not limited to, adenocarcinoma, alveolar cell carcinoma, squamous cell carcinoma, large cell and small cell carcinoma. Examples of breast carcinoma include, but are not limited to, adenocarcinoma, ductal carcinoma in situ, lobular carcinoma in situ, invasive ductal carcinoma, medullary carcinoma and mucinous carcinoma.

A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof.

Polymeric nanoparticles are a useful tool to encapsulate cytotoxins because of their controlled and sustained delivery. Among them, polymeric hydrogel nanoparticles with thermal and/or pH responsiveness are particularly attractive as vehicles for delivery and release of small molecules. Many polymeric hydrogel nanoparticles exhibit a lower critical solution temperature (LCST), which can be designed to be between about 0-35° C. The polymeric hydrogel is in a swollen/soluble state at or below room temperature, while it is in a collapsed/gel state at the physiological temperature (i.e., about 37° C.). The sol-gel transition of the hydrogel is accompanied with an apparent change of its chemical and physical properties, which could be utilized to achieve controlled release of cytotoxins encapsulated in the hydrogel. For example, cytotoxins can be effectively encapsulated in appropriately designed Pluronic hydrogel nanocapsules with minimum release (less than about 20%) for up to 2 days at a temperature above the hydrogel LCST. Nanocapsules less than about 150 nm can be easily internalized by mammalian cells via endocytosis, a natural pathway of cell self-feeding. Furthermore, the surface of the nanocapsules can be modified using ligands and/or other functional moieties such as polyethylenimine (PEI) to achieve target specific and/or facilitated intracellular delivery of cytotoxins.

In accordance with the present disclosure, a thermally responsive polymeric hydrogel nanocapsule was synthesized, characterized, and used as the vehicle for delivering cytotoxin. The nanocapsule was made of Pluronic F127 and polyethylenimine (PEI), although any suitable materials could be utilized in accordance with the present disclosure. For instance, a triblock polymer poly(ethylene oxide)-polypropylene oxide)poly(ethylene oxide), which is commercially available under the PLURONIC™ or POLOXAMER™ trade names, can be utilized to form the nanocapsule of the present disclosure. In addition, the nanocapsule can include polyethylenimine, chitosan, poly-l-lysine, or other polycations can be utilized to form the nanocapsule of the present disclosure. In certain embodiments, the nanocapsule can be formed using poly(N-isopropylacrylamide) or another amphiphilic polymer that exhibits a LCST of from about 0° C. to about 37° C., particularly from about 4° C. to about 37° C.

The nanocapsule can be loaded with one or more cytotoxins, such as the type described herein. The nanocapsule can be delivered to any suitable type of cell including mammalian cells. The temperature dependent properties (i.e., thermal responsiveness) of the nanocapsule such as size, surface charge, and particularly wall permeability can be utilized to achieve nanoencapsulation and controlled release of cytotoxins both outside and inside mammalian cancer cells.

The nanocapsules of the present disclosure can be surface-modified using polyethylene glycol or other stealth materials for in vivo drug delivery. In addition, the nanocapsules can be surface-modified using folic acid or other targeting moieties for target specific in vivo drug delivery. However, such examples are not meant to be limiting and any suitable compounds can be used to surface-modify the nanocapsules of the present disclosure.

Products and methods resulting from the present disclosure include, but are not limited to: a combined minimally invasive surgical modality of cryoablation and nanoencapsulated drug for which complete tumor destruction can be accurately monitored by intraoperative ultrasound imaging of the iceball of frozen tissue; an effective protocol for nanoencapsulation, intracellular delivery, and controlled release of cytotoxin using thermally responsive nanocapsules; and a nanoencapsulated form of cytotoxin that can minimize the drug systemic cytotoxicity and can be used to destroy cancer cells at a systemic concentration much lower than that required for a drug in its free form.

In comparison to existing technology, the products and methods of the present disclosure have numerous advantages including that the augmented cryoablation procedure ensures complete destruction of the whole frozen tumor in an iceball, making it accurate to monitor the ‘kill zone’ using intraoperative ultrasound to eliminate cancer recurrence. Additionally, release of a nanoencapsulated drug is controlled to be within the tumor, which augments cryoinjury in the tumor while minimizing systemic cytotoxicity of the drug to healthy tissue. As a result of its positively charged surface at 37° C., the nanocapsule mediated intracellular drug delivery is much more efficient than many other nanoparticles, which can significantly reduce the interstitial drug concentration required for tumor destruction and therefore the drug systemic cytotoxicity. Also, the thermal responsiveness of the nanocapsule can be used to avoid drug sequestration and degradation in the endosome/lysosome system, which can further reduce the amount of drug required for tumor destruction and its systemic cytotoxicity. Further, drugs encapsulated in the nanocapsule with negligible release at 37° C. (and eventually removed from the body by the reticuloendothelial system) are much less toxic than their form when applied systemically. The nanocapsule with a cross linked polymer surface is much more structurally stable than liposomes and their derivatives for drug delivery and nanocapsule synthesis and drug encapsulation processes can be separated, which minimizes destabilization of the drug by organic solvents, high temperature, and or pH deviation associated with direct encapsulation of drug during synthesis of liposomes or other nanoparticles. Lastly, cryoablation is a minimally invasive tissue conservation procedure that causes less discomfort (due to the analgesic effect of cold) and less cosmetic damage in comparison to, for example, mastectomy, lumpectomy and hyperthermic ablation of the breast.

Reference now will be made to exemplary embodiments of the invention set forth below. Each example is provided by way of explanation of the invention, not as a limitation of the invention.

### EXAMPLE 1

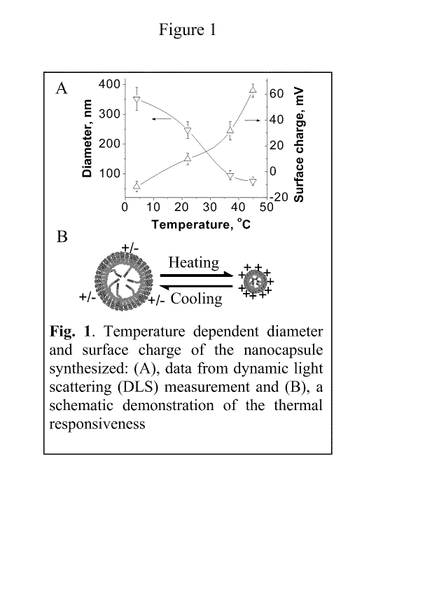

A thermally responsive empty core-shell structured nanocapsule made of Pluronic F127 (BASF Corp.) and poly(ethylenimine) (PEI) has been successfully synthesized. A broad thermal responsiveness of the Pluronic F127-PEI nanocapsule in diameter determined by dynamic light scattering analysis is shown in FIG. 1A: the nanocapsule is ˜95 nm at 37° C. whereas it is ˜352 nm at 4° C. Also shown in the figure is the temperature dependent surface charge of the nanocapsule in 1×PBS: it is much more positively charged at 37 (31.6 mV) than 22° C. (9.8 mV) and even become negatively charged at 4° C. (−11.6 mV). A schematic demonstration of the thermal responsiveness of the nanocapsule is given in FIG. 1B showing the reversible change of size and surface charge of the nanocapsule in response to heating and cooling.

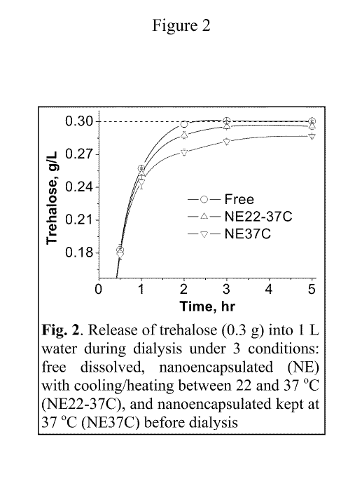

This nanocapsule has been used to physically encapsulate a small molecular weight hydrophilic trehalose (M_{w}=342 Da) for its intracellular delivery and controlled release. It was found that trehalose can be loaded into the nanocapsule by simply soaking the nanocapsule in aqueous trehalose solution (15 wt %) at 22° C. when its wall permeability is high followed by freeze dry to remove water altogether. FIG. 2 shows the release of a total of 0.3 g trehalose during dialysis into 1 L water at 37° C. under three different conditions: free trehalose in 2 ml water (Free), free and encapsulated (in 20 mg nanocapsules) trehalose in 2 ml water kept at 37° C. before dialysis (NE37° C.), and free and encapsulated (in 20 mg nanocapsules) trehalose in 2 ml water that was cooled to 22° C. for 15 minutes and heated back to 37° C. before dialysis (NE22-37° C.). Clearly, the free dissolved trehalose was completely released from the dialysis bag in ˜2 hr, while trehalose release from the other two conditions with nanoencapsulation is much slower. The release for the sample kept 37° C. (i.e., NE37C) reached equilibrium at approximately 4 hours while it is approximately 3 hours for the sample with cooling and heating between 22 and 37° C. (i.e., NE22-37C). Moreover, approximately 11.3 and 4.3 mg trehalose were still retained in the NE37C and NE22-37C samples under the equilibrium state, respectively. The amount of trehalose retained in the NE22-37C sample is much less than that in the NE37C sample, indicating a significant amount of trehalose was released from the nanocapsule during cooling at 22° C. and the subsequent heating back to 37° C. In other words, a quick release of the nanoencapsulated trehalose can be achieved by thermally cycling the trehalose loaded nanocapsule between 22 and 37° C. Altogether, it was found that a significant amount the sugar (sugar:nanocapsule=11.3:20 or 1:1.78 in weight) can be physically encapsulated in the nanocapsule after 4 hours dialysis and temperature can be used to control the release of the encapsulated sugar.

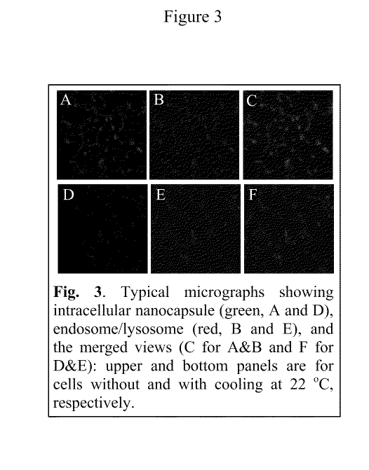

Moreover, it has been determined that a significant amount of the nanocapsule and sugar can be taken up by NIH 3T3 fibroblasts by incubating the cells with the trehalose loaded nanocapsules for as short as 40 min at 37° C. and cytotoxicity of the nanocapsules was determined to be negligible. The nanocapsules were found to be taken up by the cells via an accelerated endocytotic pathway (absorptive endocytosis) as a result of electrostatic attraction between the positively charged nanocapsules (see FIG. 1 at 37° C.) and the negatively charged cell plasma membrane. Cellular uptake of the nanocapsule was visualized by labeling the nanocapsule with a green fluorescence probe (FITC) and fixing the cells right after 40 minutes of incubation as shown in FIG. 3A. FIG. 3B which show the red fluorescence (LysoTracker Red) stain of endosome/lysosome of the fixed cells. A merged view of the green and red channels (FIG. 3C) gives a yellowish appearance indicating extensive co-staining of the two fluorescent probes. Therefore, the nanocapsules are primary sequestered in the endosome/lysosome system right after their uptake, as with many other nanoparticles. Sequestration and subsequent degradation of drug/therapeutic agents in endosome/lysosome have been suggested as the bottleneck of nanoparticle mediated cytosolic drug delivery. The challenge, however, can be overcome using the thermal responsive nanocapsule by cooling the cells to room temperature or lower. As shown in FIG. 3D, the green fluorescence becomes faint in cells loaded with FITC labeled nanocapsule after cooling at 22° C. for 15 min. Moreover, the yellowish color of the merged view of the green (FIG. 3D) and red channels (FIG. 3E) is not observable for the cells with cooling at 22° C. (FIG. 3F), indicating that the nanocapsules escape the endosome/lysosome presumably by breaking the subcellular organelles as a result of its more than 15 times of volume expansion (see FIG. 1). At 22° C., the nanocapsule (˜250 nm) is much bigger than the endosome (˜150 nm). Moreover, water can suck into the nanocapsule during cooling (volume expansion) to dissolve the encapsulated sugar and squeeze the dissolved sugar out of the nanocapsule during heating (volume contraction). Therefore, release of the encapsulated sugar from the nanocapsule into the cytosol can be achieved by thermally cycling the nanocapsule between a low temperature (room temperature or below) and 37° C., which is exactly the procedure used in cryoablation of various diseased tissues including that of cancer. Therefore, the thermally responsive nanocapsule is a natural design to achieve controlled drug delivery during a cryoablation procedure. Doxorubicin (DOX), a hydrophilic, small molecular weight (543.5 Da, similar to trehalose) anticancer drug that has been commonly used for breast cancer chemotherapy can be utilized as a substitute for trehalose.

To encapsulate DOX in thermally responsive nanocapsules for controlled delivery, the nanoencapsulation and controlled release of DOX can be achieved using a method recently developed utilizing the temperature dependent wall permeability of the nanocapsule.

The small molecular weight, hydrophilic trehalose has been successfully encapsulated in the thermally responsive nanocapsules for controlled release. This method can be used to encapsulate the small molecular weight, hydrophilic DOX. In this method, trehalose is loaded into the nanocapsule by soaking the nanocapsule in an aqueous solution of the sugar at 22° C. The soaking will be performed at 4° C. for loading DOX since the drug is slightly bigger than trehalose (543.5 vs. 342 Da). The nanocapsule at 4° C. is more swollen than at 22° C. (˜350 vs. 250 nm in diameter, FIG. 1). Therefore, the wall permeability should be higher at 4° C. than at 22° C. as well. A detailed description of the methods used is given below.

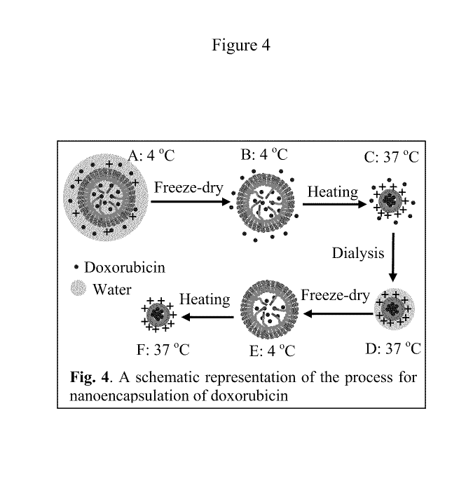

To load doxorubicin into the thermally responsive nanocapsule, the nanocapsule is soaked in the aqueous doxorubicin solutions at various concentrations (1-10 mg/ml) at 4° C. (FIG. 4A) followed by freeze drying to remove water altogether (FIG. 4B), heating to 37° C. (FIG. 4C), dialyzing at 37° C. (FIG. 4D) when the nanocapsule is shrunk with extremely low permeability to remove non-encapsulated doxorubicin, and freeze dried again to remove water altogether (FIG. 4E) for use immediately or storing at 4° C. for future use. In either case, the nanocapsule is first heated to 37° C. before any further use (FIG. 4F). The ratio of the amount of encapsulated DOX to the amount of nanocapsule is determined to evaluate the efficiency of the loading process.

The release behavior of the nanoencapsulated doxorubicin is studied under various conditions: kept at 4° C., kept at 37° C., heating and cooling between 4 and 37° C. once, and heating and cooling between 4 and 37° C. twice. The time for heating and cooling at the two temperatures will be 15 minutes, which is typically used during a cryoablation procedure. For the release studies, a total of 100 μg of encapsulated doxorubicin can be dissolved in 1 ml water in a dialysis bag (Spectro/Por, MWCO, 50 kDa) and dialyzed against a total of 1 L water. Doxorubicin concentration in the 1 L water can be sampled at various times for up to 5 days. Since doxorubicin is light sensitive, all samples can be kept in the dark by wrapping them with aluminum foil. The amount of free and encapsulated doxorubicin in a solution can be determined using a standard approach by measuring the absorbance at 490 nm using a Shimazu (Columbia, Md.) UV-2101PC spectrophotometer and comparing with a standard curve between absorbance and the known amount of DOX.

As with what has been previously discovered by the present inventors for trehalose, DOX can be effectively encapsulated in the thermally responsive nanocapsules using the procedure given in FIG. 4. It is further believed that DOX can be retained in the nanocapsule at 37° C. for days with negligible release since the drug is bigger than trehalose that has been shown to be effectively trapped in the nanocapsule at 37° C. However, a quick release of the nanoencapsulated drug is expected by heating and cooling the drug-loaded nanocapsules between 4 and 37° C., due to the more than 50 times of volume change (FIG. 1) to such water into the nanocapsule during cooling and squeezed the dissolved DOX out of the nanocapsule during heating.

To determine the combined effect in vitro of freezing and nanoencapsulated DOX on destroying breast cancer cells, breast cancer cells can be incubated with the nanoencapsulated DOX and/or cooled to temperatures between −40 and 4° C. The cells can be further cultured for two days to check their viability and determine the combined effect of DOX and freezing in vitro.

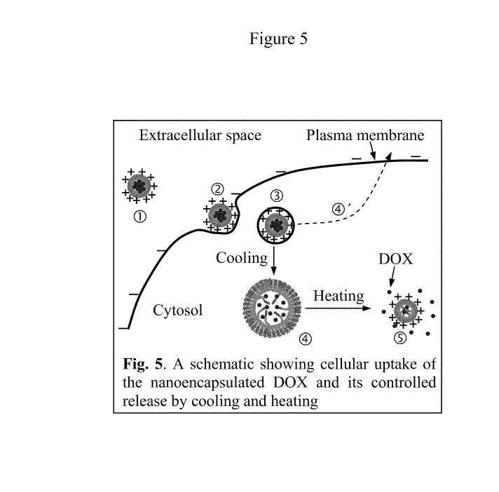

A number of anticancer drugs including peplomycin, 5-fluorouracil, and bleomycin at a concentration that would not kill cancer cells alone have been shown to significantly enhance cryoinjury in several cancer cell lines. DOX, an anticancer drug that has been commonly used for chemotherapy of breast cancer, is therefore believed to have a synergistic effect with freezing on destroying breast cancer cells as well, particularly when it is delivered into the cells using the thermally responsive nanocapsule. The positively charged nanocapsule can significantly facilitate cellular uptake of DOX via an accelerated endocytotic pathway that is much faster (40 min) than cellular uptake of free DOX dissolved in extracellular medium (i.e., pinocytosis, hours to days). A schematic representation of the nanocapsule mediated cellular uptake of DOX and its controlled release into the cytosol is given in FIG. 5. The positively charged DOX-loaded nanocapsule floating in culture medium at 37° C. (\textcircled{1}) is attracted onto the negatively charged plasma membrane and enwrapped in a clathrin-coated pit on the plasma membrane (\textcircled{2}). The coated pit then buds into the cytoplasm to form the early endosome (˜150 nm in size, \textcircled{3}). If kept at 37° C., the nanocapsule will be sequestered in the endosome/lysosome system followed by potential exocytosis (\textcircled{4}′). However, a quick release of the nanoencapsulated DOX into the cytosol can be achieved by cooling the cells to 4° C. so that the nanocapsule can break the early endosome as a result of its more than 50 times volume expansion (\textcircled{4}), the nanocapsule is ˜350 and 95 nm in diameter at 4 and 37° C., respectively according to FIG. 1), followed by heating the cells back to 37° C. to squeeze the dissolved trehalose out of the nanocapsule as a result of the more than 50 times of volume contraction (\textcircled{5}). Therefore, the cooling/heating dependent release of DOX into cytosol can provide a means to destroy diseased cells in a controllable manner.

MCF-7 cells can be maintained in DMEM (Invitrogen, Carlsbad, Calif.) supplemented with 5% fetal bovine serum, 2 mM L-glutamine, 100 U/ml penicillin/streptomycin, and 0.01 mg/ml insulin at 37° C. in a 5% CO₂ incubator. Only MCF-7 cells in their log phase of growth (˜80% confluency) are typically used. Cells will be detached with 0.05% trypsin/EDTA and collected by centrifugation at 180×g.

A total of 0.5 ml nanoencapsulated DOX at a concentration from 0.1 to 100 μg/ml can be incubated with 1×10⁵ MCF-7 human breast cancer cells (ATCC, Manassas, Va.) cultured in 33 mm Petri dish at 37° C. for 40 min. The cells can then be washed twice with warm (37° C.) 1×PBS followed by either putting in culture immediately or after treated with a double-cycle cooling (at −5° C./min to −40, −20, −10, −5, and 4° C. and hold for 10 minutes) and heating (at 5° C./min to 37° C. and hold for 10 minutes) protocol. The double-cycle cooling and heating procedure is typically used for tissue cryoablation and can result in a quick release of DOX from the nanocapsule into cytosol. Fresh cells seeded at the same density without any treatment can be studied to serve as control. For all samples, the cells will be further cultured for 2 days and cell viability will then be assessed using the well established MTT assay.

The thermally responsive nanocapsule mediated intracellular delivery of DOX can provide a way for selective destruction of cancer cells: only cells taken up the drug loaded nanocapsule with the cooling (at 4° C. or lower) and heating treatments will be killed. It is also believed that the nanocapsule mediated intracellular delivery of DOX will significantly decrease the extracellular concentration of the drug required for destruction of MCF-7 cells. By combining nanoencapsulated DOX with freezing, it is believed that the threshold temperature for breast cancer destruction can be improved to that on to the edge of an iceball (˜−5° C.) during cryoablation.

The present disclosure can also be utilized to determine the combined effect in vivo of freezing and nanoencapsulated DOX on destroying breast cancer tumor.

Breast cancer tumor grown subcutaneous in mice can be treated with the nanoencapsulated DOX and/or cryoablation. Tumor volume after the treatment can be monitored to determine the combined effect of DOX and freezing in vivo.

A major challenge of cryoablation is to destroy tumor tissue while sparing as much as possible the adjacent normal tissue. This difficulty can be alleviated by intravenous injection of doxorubicin encapsulated in the thermally responsive nanocapsules. The small (<100 nm at 37° C.) DOX loaded nanocapsules are believed to selectively accumulate in the tumor space because of the enhanced permeability and retention (EPR) of tumor tissue resulting from its leaky vasculature and lack of lymphatic drainage. Moreover, the small size of the nanocapsules at 37° C. can minimize their removal from the blood stream by the reticuloendothelial system (RES) to extend their circulating time for being taken up by tumor tissue. The tumor can then be selectively destroyed by localized freezing/heating using a cryoprobe as a result of both freezing and DOX released into the cytosol in response to the temperature variation during cryoablation.

One million MCF-7 cells suspended in 30 μl of diluted Matrigel (BD biosciences, diluted 4 times) can be inoculated subcutaneously into the hind limb of male athymic nude mice weighing 20 to 25 g purchased from the National Cancer Institute. The tumor can be allowed to grow to a diameter of ˜1 cm in 6 to 8 weeks. The 1 cm tumor can be treated in situ under the following conditions: control (no treatment), cryoablation alone, DOX alone, and DOX combined with cryoablation. Nanoencapsulated DOX of 0.5-5 mg per kg body weight dissolved in 30 μl saline at 37° C. can be injected intravenously into the mouse tail vein. Cryoablation can be done using a tabletop argon based cryoablation system is known for cryoablation of breast tumors in patients. A double freezing/heating protocol that is typically used in cryoablation of breast cancer in patients can be adopted. In each cycle, freezing will be stopped immediately once the iceball covers the whole tumor volume. The tissue can be actively thawed by turning on the heating mechanism of the cryoprobe during the first cycle and the thawing is passive for the send cycle. For studying the combined effect of DOX and cryoablation, the latter will be done 45 minutes after DOX injections to allow cellular uptake of the nanoencapsulated DOX accumulated in the tumor (particularly on its peripheral). During cryoablation, the animal is anesthetized by intraperitoneal injection of ketamine and xylazine at 10 and 1 mg/kg body weight, respectively. Volume of the tumor treated under the various conditions can be monitored for 1 month using a standard monitoring methods as described in Corbett, T. H., D. P. Griswold, Jr., B. J. Roberts, J. C. Peckham, and F. M. Schabel, Jr; Biology and therapeutic response of a mouse mammary adenocarcinoma (16/C) and its potential as a model for surgical adjuvant chemotherapy; Cancer Treat Rep, 1978. 62(10): p. 1471-88, incorporated by reference herein.

Tumor recurrence with cryoablation alone is expected due to the incomplete destruction of tumor cells inside the iceball next to its edge. However, complete destruction of the tumor is expected for the combined treatment of encapsulated DOX and cryoablation, even at a dose of the nanoencapsulated DOX that is insufficient to destroy tumor alone.

What is claimed:

1. A method for intracellular delivery of cytotoxin in combination with cyoablation comprising:
encapsulation of one or more cytotoxins in a thermally responsive nanocapsule by decreasing the temperature of the nanocapsule to increase the permeability of the nanocapsule whereby the one or more cytotoxins are sucked into or diffuse into the nanocapsule;
increasing the temperature of the nanocapsule and delivering the nanocapsule into a cell; and
performing cryoablation in proximity to the cell resulting in the release of the one or more cytotoxins from the nanocapsule into the cell.

2. The method of claim 1, wherein the nanocapsule comprises a polymeric nanoparticle.

3. The method of claim 1, wherein the nanocapsule comprises a polycation comprising polyethylenimine, chitosan, or poly-l-lysine.

4. The method of claim 1, wherein the nanocapsule has a diameter of less than 150 nm at a temperature of greater than 35° C.

5. The method of claim 1, wherein the nanocapsule has a diameter of greater than 150 nm at a temperature of less than 25° C.

6. The method of claim 1, wherein the temperature is decreased to less than about 25° C.

7. The method of claim 1, wherein the temperature is increased to greater than about 35° C.

8. The method of claim 1, wherein the nanocapsule is delivered into the cell by endocytosis and is located in an endosome of the cell.

9. The method of claim 8, wherein the cryoablation decreases the nanocapsule temperature after it is delivered into the cell, thereby increasing the diameter of the nanocapsule so as to cause damage to the endosome of the cell whereby the nanocapsule is released into the cytosol of the cell.

10. The method of claim 1, further comprising further increasing the nanocapsule temperature after it is delivered into the cell, thereby decreasing the diameter of the nanocapsule whereby the one or more cytotoxins are squeezed out of the nanocapsule into the cell.

11. The method of claim 1, wherein the cell comprises a cancer cell.

12. The method of claim 1, wherein the one or more cytotoxins comprise taxol, cytochalasin B, gramicidin D, ethidium bromide, emetine, mitomycin, etoposide, tenoposide, vincristine, vinblastine, colchicin, doxorubicin, paclitaxel, daunorubicin, dihydroxy anthracin dione, mitoxantrone, mithramycin, actinomycin D, 1 dehydrotestosterone, glucocorticoids, procaine, tetracaine, lidocaine, propranolol, puromycin, analogs or homologs thereof, or combinations thereof.

13. The method of claim 1, wherein the one or more cytotoxins comprise doxorubicin.

14. The method of claim 1, wherein the nanocapsule has a positive surface charge.

15. A thermally responsive nanocapsule comprising:
a polymeric nanocapsule comprising a shell and a core, the shell having a diameter of greater than 150 nm at a temperature of less than 25° C. and a diameter of less than 150 nm at a temperature of greater than 25° C., the core comprising one or more cytotoxins.

16. The nanocapsule of claim 15, wherein the nanocapsule comprises a polycation comprising polyethylenimine, chitosan, or poly-l-lysine.

17. The nanocapsule of claim 15, wherein the nanocapsule comprises a poloxamer, an amphiphilic polymer, or combinations thereof.

18. The nanocapsule of claim 17, wherein the amphiphilic polymer comprises poly(N-isopropylacrylamide).

19. The nanocapsule of claim 17, wherein the amphiphilic polymer exhibits a lower critical solution temperature between about 0° C. and about 37° C.

20. The nanocapsule of claim 15, wherein the one or more cytotoxins comprise taxol, cytochalasin B, gramicidin D, ethidium bromide, emetine, mitomycin, etoposide, tenoposide, vincristine, vinblastine, colchicin, doxorubicin, paclitaxel, daunorubicin, dihydroxy anthracin dione, mitoxantrone, mithramycin, actinomycin D, 1 dehydrotestosterone, glucocorticoids, procaine, tetracaine, lidocaine, propranolol, puromycin, analogs or homologs thereof, or combinations thereof.
